(19) United States
(12) Patent Application Publication
(10) Pub. No.: US 2007/0123500 A1
(43) Pub. Date: May 31, 2007

(54) Prodrugs of ERbeta-selective substances, processes for their preparation and pharmaceutical compositions comprising these compounds

(76) Inventors: Gerd Mueller, Jena (DE); Dirk Kosemund, Erfurt (DE); Ralf Wyrwa, Rothenstein (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/604,891

(22) Filed: Nov. 28, 2006

Related U.S. Application Data
(60) Provisional application No. 60/742,524, filed on Dec. 6, 2005.

(30) Foreign Application Priority Data
Nov. 29, 2005 (DE) 102005057224.3

(51) Int. Cl. A61K 31/56; C07J 1/00

(52) U.S. Cl. 514/182; 552/626

(57) ABSTRACT

The present invention makes available prodrugs of 9α-substituted oestratrienes of the general formula (I) in which the group Z is bonded to the steroid, 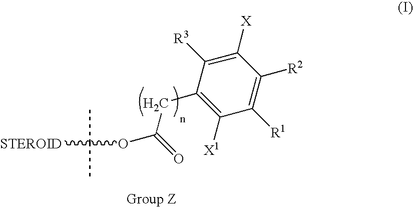 processes for their preparation, pharmaceutical compositions which comprise these compounds and use thereof. The compounds of the general formula I according to the invention do not bind to the oestrogen receptor a and/or β. They bind to carboanhydrases and inhibit these enzymes.

The invention relates to prodrugs of ERβ-selective substances of the general formula (I), 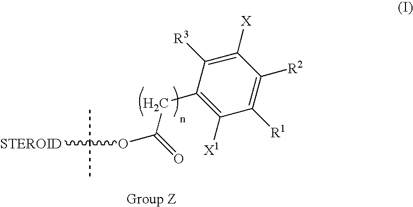
a process for their preparation, pharmaceutical compositions comprising these compounds and their use for the production of medicaments.

Oestrogens play an important role in the body in both sexes, In the maturing body, oestrogens are involved in the imprinting of sex characteristics. In both sexes, oestrogens control the changes in the body during pubescence, such as the sudden increase in growth and subsequently the ending of bone growth. In all phases of life, oestrogens play a central role in bone metabolism in both sexes (1, 4). Their loss leads to the breakdown of osseous tissue and involves the risk of increased brittleness of the bone.

In women, the oestrogens secreted by the ovary dominate in the body. In pregnancy, the placenta forms large amounts of oestrogen. In men, oestrogens are mainly formed “peripherally” by the aromatization of testosterone or of the adrenal androgens in various end organs, such as the CNS, the bone or the intestinal epithelium. This adjustment permits the physiological effects of oestrogen in men at very low oestradiol levels in the blood. In men and women with a genetic defect of aromatase or of the oestrogen receptor, the bone is massively perturbed with respect to growth and maintenance (2).

Whereas for natural oestrogens oral administration (10) is problematical due to their low oral bioavailability, conventional chemically modified oestrogens having improved bioavailability (for example ethynyl-oestradiol) often have the disadvantage of causing a markedly increased oestrogen effect in the liver (3, 9, 10). This hepatic oestrogenicity concerns a number of functions, such as transport proteins, lipid metabolism, blood pressure regulation and clotting factors (5, 7, 11, 12, 14). The secretion of IGF-I (8), particularly important for the maintenance of musculature and bone, is also adversely affected by hepatic effects of oestrogen (12, 13, 6).

In WO 01/77139, novel 8β-substituted oestratrienes are described, where the 8β substituent can be a straight- or branched-chain, optionally partially or completely halogenated alkyl or alkenyl radical having up to 5 carbon atoms, an ethynyl or propyn-1-yl radical, which as pharmaceutical active compounds show a higher in vitro affinity to oestrogen receptor preparations of rat prostate than to oestrogen receptor preparations of rat uterus and in vivo exhibit a preferential action on bone in comparison to the uterus and/or marked action with respect to stimulation of the expression of 5HT2a receptor and transporter. These compounds can preferably be used for the treatment of diseases which are caused by an oestrogen deficit.

WO 03/104253 describes novel 9α-substituted oestra-trienes having a straight- or branched-chain, optionally partially or completely halogenated alkenyl radical having up to 6 carbon atoms, an ethynyl or propyn-1-yl radical in position 9α, which likewise show a higher in vitro affinity to oestrogen receptor preparations of rat prostate than to oestrogen receptor preparations of rat uterus and in vivo preferably exhibit a preferential action on the ovary in comparison to the uterus. These compounds can preferably be used for the treatment of diseases which are caused by an oestrogen deficit.

From WO 01/91797, steroidal compounds are known which are bonded to erythrocytes via a group —SO₂NR¹R² and accumulate there. The concentration ratio of the compounds between erythrocytes and plasma is 10-1000:1, preferentially 30-1000:1, such that we can speak of depot formation in the erythrocytes. Owing to the strong bonding of the compounds to the erythrocytes, metabolization during the liver passage is avoided. Disadvantageously, despite reduced metabolization using the dosages indicated, therapy-relevant active compound levels are not afforded.

It is therefore the object of the present invention to make available prodrugs of ERβ-selective compounds, which make the ERβ-selective compounds orally bioavailable.

This object is achieved by sulphamoyl compounds of 9α-substituted oestratrienes of the general formula (I), in which the group Z is bonded to the steroid to be released 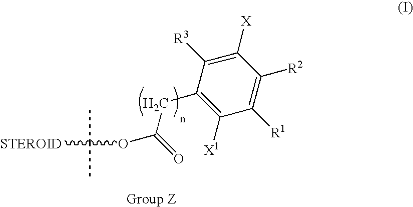
in which n is a number 0-4,
R¹ is a radical —SO₂NH₂ or —NHSO₂NH₂,
   where R², R³ and X, X¹ independently of one another are a hydrogen atom, a halogen atom, a nitrile group, a nitro group, a C₁₋₅-alkyl group, a CₚF₂ₚ₊group with p=1−3, a group OC(O)—R²⁰, COR²⁰, COOR²⁰,OR²⁰,C(O)NHR²⁰ or OC(O)NH—R²¹,
   where R²⁰ and R²¹ are a C₁₋₅-alkyl group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
   R²⁰ can moreover be a hydrogen, or
R² is a radical —SO₂NH₂ or —NHSO₂NH₂,
   where R¹, R³ and X, X¹ independently of one another are a hydrogen atom, a halogen atom, a nitrile group, a nitro group, a C₁₋₅-alkyl group, a CₚF₂ₚ₊₁ group with p=1−3, a group OC(O)—R²⁰, COOR²⁰, OR²⁰, C(O)NHR²⁰ or OC(0)NH—R²¹,
   where R²⁰ and R²¹ are a C₁₋₅-alky group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
   R²⁰ can moreover be a hydrogen, or
R³ is a radical —SO₂NH₂ or —NHSO₂NH₂,
   where R¹, R² and X, X¹ independently of one another are a hydrogen atom, a halogen atom, a nitrile group, a nitro group, a C₁₋₅-alkyl group, a CₚF₂ₚ₊₁ group with p=1−3, a group OC(O) —R²⁰, COOR²⁰, OR²⁰, C(O)NR²⁰ or OC(O)NH—R²⁰,
   where R²⁰ and R²¹ are a C₁₋₅-alkyl group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
   R²⁰ can moreover be a hydrogen, and

STEROID is a steroidal ABCD ring system of the formula (A): 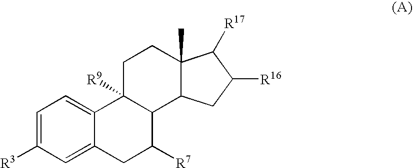
where the radicals R⁷, R⁹, R¹⁶ and R¹⁷ have the following meaning:
R³ is Z and
R¹⁶ is an OH group, a tri(C₁₋₄-alkyl)silyloxy group or a group OC(O)—R²⁰, or
R³ is OH, OMe, a tri(C₁₋₄-alkyl)sily1oxy group, a group OC(O)—R2⁰ and
R¹⁶ is Z and
R⁷ is a hydrogen atom or fluorine atom, a methyl radical or ethyl radical,
R⁹ is a branched or straight-chain, optionally partially or completely halogenated alkyl, alkenyl or alkynyl radical having up to 3 carbon atoms,
R¹⁷ is a hydrogen atom or a halogen atom
   where the substituents R⁷, R¹⁶ and R¹⁷ can in each case be both in the α-position and in the β-position,
   and their pharmaceutically acceptable salts.

Furthermore, the present invention comprises the novel compounds as pharmaceutical active compounds, their preparation, their therapeutic application and pharmaceutical administration forms which contain the novel substances.

The invention relates to oestrogen derivatives which cannot bind to the oestrogen receptor themselves and from which the parent oestrogen contained is released in the body, to processes for their preparation and to pharmaceutical compositions comprising these compounds. The compounds according to the invention are prodrugs which release an ERβ-selective oestrogen (parent oestrogen) after hydrolysis of the ester group Z.

As a result of absolutely and relatively strongly attenuated actions on the ER α, undesired oestrogen effects of any classical oestrogen therapy on the uterus, the mammary gland and the liver, as are typical of undissociated oestrogens, are avoided. The compounds according to the invention have therapeutically favourable oestrogenic activities, if they are mediated by means of the ER β, in particular in the central nervous system, in the circulatory system and in the bone.

The substances according to the invention are preferably employed for oral therapy. Compared to their parent oestrogens, the compounds according to the invention have a markedly increased oral bioavailability and an increased systemic oestrogenicity, but as a rule a reduced hepatic oestrogenicity. As a result of this dissociation of desired and undesired hormonal effects, medicaments which at the same time are therapeutically more efficacious and better tolerable in comparison to the prior art are made possible.

The substances according to the invention are cleaved enzymatically or hydrolytically in the body, no steroid sulphatases (STS) being needed, such as, for example, for the cleavage of oestradiol 3-sulphamate. Thus the inhibition of the steroid sulphatase typical of oestrogen 3-sulphamates and disadvantageous for the achievement of strong oestrogenic effects, which is typical of oestrogen sulphamates in humans, can also be avoided. In the case of oral therapy with natural oestrogens (oestradiol, oestradiol valerate, oestrone sulfate, conjugated oestrogens), but also in the case of that with oestradiol sulphamate, high levels of oestrone dominate in the blood (10). Other than in the cycle, the concentrations of oestradiol in the blood are lower than those of oestrone. This is therefore disadvantageous, because oestrone is a more weakly active oestrogen than oestradiol.

An advantage of the substances according to the invention in comparison to those in the prior art is the preferable release of the respective parent oestrogen, that is instead of the inactive oestrone derivatives, for example 9α-ethyloestra-3,l6α-diol, 9α-methyloestra-3,16α-diol, 9α-vinyloestra-3,16α-diol and 9α-difluorovinyloestra-3,16α-diol and their 17β-fluorinated analogues.

The compounds of the general formula (I) according to the invention or their pharmaceutically acceptable salts can be employed as an individual component in pharmaceutical preparations or in combination, in particular with anti-oestrogens or gestagens. Combination with ERα-selective anti-oestrogens or with anti-oestrogens which are peripherally selectively active, i.e. which do not cross the blood-brain barrier, is particularly preferred.

A therapeutic product comprising an oestrogen and a pure anti-oestrogen for simultaneous, sequential or separate use for selective oestrogen therapy of peri- or postmenopausal conditions is already described in EP-A 0 346 014.

The substances and the pharmaceuticals comprising them are particularly suitable for the treatment of peri- and postmenopausal complaints, in particular hot flushes, sleep disorders, irritability, mood fluctuations, incontinence, vaginal atrophy, hormone deficiency-related emotional disturbances. Likewise, the substances are suitable for hormone substitution and the therapy of hormone deficiency-related complaints in the case of ovarian dysfunction caused surgically, medicinally or in another way. This also includes the prevention of loss of bone mass in postmenopausal women and andropausal men, in hysterectomized women or in women who have been treated with LHRH antagonists or agonists.

The prodrugs according to the invention of the ERβ-selective agonists can be used on their own or in combination with anti-oestrogens, aromatase inhibitors or selective estrogen receptor modulators (SERM) for the treatment of prostate hyperplasia in order to avoid oestrogen deprivation or in order to reduce its effects.

The anti-oestrogen used is preferably 7α-[9-[(4,4,5,5, 5-pentafluoropentyl)sulphinyl]nonyl]oestra-1,3,5(10)-triene-3,17β-diol (fulvestrant).

Possible aromatase inhibitors to be used are the following: anastrozole, atamestane, fadrozole, formestane, letrozole.

Possible SERM are compounds selected from the following group: raloxifen, tamoxifen, 5-(4-{5-[(RS)-(4,4,5,5,5-pentafluoropentyl)sulphinyl)pentyl}phenyl)-6-phenyl-8,9-dihydro-7H-benzocyclohepten-2-ol (WO 00/03979).

The compounds are also suitable for the alleviation of the symptoms of the andropause and menopause, i.e. for male and female hormone replacement therapy (HRT), namely both for prophylaxis and for treatment, furthermore for the treatment of symptoms accompanying dysmenorrhoea and for the treatment of acne.

The substances can moreover be employed for prophylaxis of hormone deficiency-related loss of bone mass and osteoporosis, for the prevention of cardiovascular diseases, in particular vascular diseases such as atherosclerosis, for inhibition of the proliferation of arterial smooth muscle cells, and for the treatment of primary pulmonary hypertension.

Furthermore, the substances can be employed for the treatment of inflammatory diseases and diseases of the immune system, in particular autoimmune diseases such as, for example, rheumatoid arthritis, multiple sclerosis, Crohn's disease or endometriosis.

The compounds can in particular be used for the treatment of arthritic symptoms after therapies which lead to oestrogen deprivation, for example after treatment with aromatase inhibitors or GnRH antagonists or agonists.

Moreover, the compounds can be used for the treatment of male fertility disorders and prostatic disorders. The compounds according to the invention are suitable for oestrogen treatment of carcinoma of the prostate.
The compounds can also be employed in combination with the natural vitamin D3 or with calcitriol analogues for osteogenesis or as a supportive therapy for therapies which cause loss of bone mass (for example therapy with glucocorticoids, aromatase inhibitors, GnRH agonists or antagonists, chemotherapy).

Finally, the compounds of the general formula (I) can be used in combination with progesterone receptor modulators, for example mesoprogestins such as asoprisnil, namely in particular for use in hormone replacement therapy and for the treatment of gynaecological disorders.

The compounds according to the invention as set forth in general formula (I) can moreover be used for the treatment of alopecia caused, for example, by chemotherapy.

“C₁₋₅-Alkyl group” is understood in the sense of the present invention as meaning a branched or straight-chain alkyl radical having up to 5 carbon atoms, which can be substituted, for example, by halogens such as fluorine, chlorine or bromine, OH or CN. Examples which may be mentioned are methyl, ethyl, n-propyl, i-propyl, n-butyl, i-butyl, tert-butyl or n-pentyl.

The abovementioned “C₃₋₈-cycloalkyl group” is, according to the invention, a mono- or bicyclic group which can be substituted, for example by halogens such as fluorine, chlorine or bromine, OH or CN, such as, for example, a cyclopropyl, cyclobutyl, cyclopentyl, cyclohexyl or a hydroxycyclohexyl group. The term “₁₋₄-alkylenearyl group” is understood in the sense of the present application as meaning a substituted or unsubstituted aryl radical having 6 to 15 carbon atoms, for example a phenyl group, a substituted phenyl group, such as a halophenyl group or a nitrophenyl group, or a naphthyl group.

The term “C₁₋₄-alkylenearyl group” is understood in the sense of the present application as meaning a disubstituted alkyl radical which is substituted at least by one aryl radical. Both radicals together have 7 to 15 carbon atoms, where the aryl radical can carry further constituents, such as, for example, a halogen atom. Examples are a benzyl group or a halobenzyl group.

The term “C₁₋₄-alkylene-C₃₋₈-cycloalkyl group” is understood in the sense of the present application as meaning a disubstituted alkyl radical which is substituted at least by one C₃₋₈-cycloalkyl radical. Both radicals together have 4 to 12 carbon atoms, where the cycloalkyl radical can carry further substituents, such as, for example, a halogen atom. Examples are a cyclopentylethyl, cyclohexylmethyl or cyclohexylethyl group.

The term “C₃₋₈-cycloalkylene-C₁₋₄-alkyl group” in the sense of the present application is understood as meaning a disubstituted C₃₋₈-cycloalkylene radical which is at least substituted by one C₁₋₄-alkyl radical. Both radicals together have 4 to 12 carbon atoms, where the group can carry further substituents, such as, for example, a halogen atom. Examples are a propyl-cyclohexyl or butylcyclohexyl group.

A trialkylsilyloxy group is, for example, a trimethyl-silyloxy or tert-butyldimethylsilyloxy group.

The term “halogen atom” is understood in the context of the present invention as meaning a fluorine, chlorine, bromine or iodine atom. Fluorine, chlorine and bromine are preferred.

The number “n” is preferably 0, 1 or 2.

R¹ is preferably a group —SO₂NH₂, where R², R³, X¹ and X independently of one another are preferably an H, F or Cl atom, an OH group or a methoxy group.

R² is preferably a group —SO₂NH₂, where R¹, R³, X¹ and X independently of one another are preferably an H, F or Cl atom, an OH group or a methoxy group.

R³ is preferably a group —SO₂NH₂, where R¹, R², X¹ and X independently of one another are preferably an H, F or Cl atom, an OH group or a methoxy group.

X¹ is preferably an H atom.

R⁷ is a hydrogen or a fluorine atom or a methyl radical.

R⁹ is preferably methyl, ethyl, vinyl, difluorovinyl, ethynyl or prop-1-ynyl. Ethyl, vinyl or difluorovinyl are particularly preferred for R⁹.

R³ is preferably OH, OMe, a trimethylsilyloxy or tert-butyldimethylsilyloxy radical, a benzoate, a sulphamoylbenzoate, acetate, propionate, valerate, butcyclate or cyclopentylpropionate radical.

R¹⁷ is preferably a hydrogen atom or a fluorine atom.

Particularly preferred compounds in the sense of the invention are listed below:
1) 3-hydroxy-9α-vinyloestra-1,3,5(10)-trien-16α-yl 3′-sulphamoylbenzoate,
2) 3-hydroxy-17β-fluoro-9α-vinyloestra-1,3,5(10)-trien-16α-yl 3′-sulphamoylbenzoate,
3) 3-hydroxy-7α-methyl-9α-vinyloestra-1, 3,5(10)-trien-16α-yl 3′-sulphamoylbenzoate,
4) 3-hydroxy-7α-fluoro-9α-vinyloestra-1,3,5 (10)-trien-16α-yl 3′-sulphamoylbenzoate,
5) 3-acetoxy-9α-vinyloestra-1,3,5(10)-trien-16α-yl 3′-sulphamoylbenzoate,
6) 3-acetoxy-17β-fluoro-9α-vinyloestra-1,3,5(10)-trien-16α-yl 3′-sulphamoylbenzoate,
7) 3-acetoxy-7α-methyl-9α-vinyloestra-1,3,5(10)-trien-16α-yl 3′-sulphamoylbenzoate,
8) 3-acetoxy-7α-fluoro-9α-vinyloestra-1,3,5(10)-trien-16α-yl 3′-sulphamoylbenzoate,
9) 3-hydroxy-9α-vinyloestra-1,3,5(10)-trien-16α-yl 2′-chloro-5′-suiphamoylbenzoate,
10) 3-hydroxy-17β-fluoro-9α-vinyloestra-1,3,5(10)-trien-16α-yl 2′-chloro-5′-sulphamoylbenzoate,
11) 3-hydroxy-7α-methyl-9α-vinyloestra-1,3,5(10)-trien-16α-yl 2′-chloro-5′-sulphamoylbenzoate,
12) 3-hydroxy-7α-fluoro-9α-vinyloestra-1,3,5(10)-trien-16α-yl 2′-chloro-5′-sulphamoylbenzoate,
13) 3-acetoxy-9α-vinyloestra-1,3,5(10)-trien-16α-yl 2′-chloro-5′-sulphamoylbenzoate,
14) 3-acetoxy-17β-fluoro-9α-vinyloestra-1,3,5(10)-trien-16α-yl 2′-chloro-5′-sulphamoylbenzoate,
15) 3-acetoxy-7α-methyl-9α-vinyloestra-1,3,5(10)-trien-16α-yl 2′-chloro-5′-sulphamoylbenzoate,
16) 3-acetoxy-7α-fluoro-9α-vinyloestra-1,3,5(10)-trien-16α-yl 2′-chloro-5′-sulphamoylbenzoate,
17) 3-hydroxy-9α-vinyloestra-1,3,5(10)-trien-16α-yl 4′-sulphamoylbenzoate,
18) 3-hydroxy-17β-fluoro-9α-vinyloestra-1,3,5(10)-trien-16α-yl 4′-sulphamoylbenzoate,
19) 3-hydroxy-7α-methyl-9α-vinyloestra-1,3,5(10)-trien-16α-yl 4′-sulphamoylbenzoate,
20) 3-hydroxy-7α-fluoro-9α-vinyloestra-1,3,5(10)-trien-16α-yl 4′-sulphamoylbenzoate,
21) 3-acetoxy-9α-vinyloestra-1,3,5(10)-trien-16α-yl 4′-sulphamoylbenzoate,
22) 3-acetoxy-17β-fluoro-9α-vinyloestra-1,3,5(10)-trien-16α-yl 4′-sulphamoylbenzoate,
23) 3-acetoxy-7α-methyl-9α-vinyloestra-1,3,5(10)-trien-16α-yl 4′-sulphamoylbenzoate,
24) 3-acetoxy-7α-fluoro-9α-vinyloestra-1,3,5(10)-trien-16α-yl 4′-sulphamoylbenzoate

### IN VITRO EXPERIMENTS

a) Blood-plasma Concentration Ratio—Test Principle and Experimental Description:

The SO₂—NH₂ group of the substances according to the invention can lead to a concentration in erythrocytes as a result of binding to carboanhydrases.

Test Principle:

Freshly obtained, heparinized blood from a rat is treated with a defined amount of active compound. The active compound concentration in the plasma obtained therefrom is measured against a calibration curve of spiked (with a known active compound concentration) plasma. The blood-plasma ratio is calculated from the measured concentration and the theoretical concentration.

In contrast to the results published in WO 01/91797, the concentration ratios of the compounds according to the invention between erythrocytes and plasma are not in a range from 10-1000:1, but in the range <10:1. The compound 17β-fluoro-3-hydroxy-9α-vinyloestra-1,3,5(10)-trien-16α-yl 3′-sulphamoylbenzoate accumulates with a blood/plasma ratio of approximately 3.7 in the rat and in the human erythrocytes at 1.2.

b) Carboanhydrase Inhibition—Test Principle and Experimental Description:

Photometric determination of the inhibition of human carboanhydrase I or II by sulphonamides or sulphamates on microtitre plates with the aid of the enzymatic conversion of nitrophenyl acetate with a colour change from colourless to yellow.

TABLE 1
IC₅₀ inhibitory values of human carboanhydrase I and II

| | CAI | | CAII | |
|---|---|---|---|---|
| | | IC50 (nM) | | IC50 (nM) |
| Inhibitor | IC50 (nM) | literature | IC50 (nM) | literature |
| Oestradiol 3- | 157 ± 10.6 | — | 21.6 ± 1.5 | — |
| sulphamate | | | | |
| 17β-Fluoro-3- | 3700 | — | 720 | — |
| hydroxy-9α- | | | | |
| vinyloestra- | | | | |
| 1,3,5(10)- | | | | |
| trien-16α-yl | | | | |
| 3′-sulphamoyl- | | | | |
| benzoate | | | | |
| 17β-Fluoro-3- | >10 000 | — | >10 000 | — |
| hydroxy-9α- | | | | |
| vinyloestra- | | | | |
| 1,3,5(10)- | | | | |
| trien-16α-yl | | | | |
| benzoate | | | | |
| 3-Hydroxy-9α- | 3400 | — | 490 | — |
| vinyloestra- | | | | |
| 1,3,5(10)- | | | | |
| trien-16α-yl | | | | |
| 3′-sulphamoyl- | | | | |
| benzoate | | | | |
| Acetazolamide | 1200 | 1900 | 60 | 90¹ |
| (known CA | | | | |
| inhibitor) | | | | |

¹Literature: C. Landolfi, M. Marchetti, G. Ciocchi, and C. Milanese, Journal of Pharmacological and Toxicological Methods 38, 169-172 (1997).

Despite the low blood-plasma concentration ratio, in all cases binding (inhibition) to the two isoenzymes carboanhydrase CA I and CA II could be shown in the erythrocytes. Of importance for the properties as an oestrogen is the binding to erythrocytes induced by affinity to the carboanhydrases. This binding is essential for a reduced extraction of the orally administered substance in the first liver passage. A high or lower affinity to the erythrocytic carboanhydrases, more rapid or delayed release from this depot and subsequent hydrolysis determine the therapeutic employability of the substances according to the invention. The compounds according to the invention thus open up the possibility on equimolar substance administration of achieving higher short-lasting or more uniform low and longer-lasting hormone levels. The potency and duration of action are thus varied and a therapy tailored to the organism is made possible.

### IN VIVO EXPERIMENTS

I.V./P.O. Pharmacokinetics

For the investigation of pharmacokinetic properties and parameters, rats were administered i.v. and p.o. and blood samples were obtained for determination at various points in time.

Test principle:

The various pharmacokinetic parameters can be determined by means of the time profile which the substance shows, and with the aid of appropriate pharmacological software. The concentration of the test substance in the serum or plasma samples was determined by HPLC-UV or by LCMS/MS.

By means of the recovery of the substance per point in time the breakdown of the active compound in the organism can be shown. The rate of breakdown serves for the calculation of the individual pharmacokinetic parameters.

Investigations of the i.v./p.o. kinetics on the rat showed that 17β-fluoro-9α-vinyloestra-1,3,5(10)-triene-3,16-α-diol after release form the prodrug 17β-fluoro-3-hydroxy-9α-vinyloestra-1,3,5(10)-trien-16α-yl 3-sulphamoylbenzoate was bioavailable to 17%. After administration of the “parent oestrogen” 17β-fluoro-9α-vinyloestra-1,3,5(10)-triene-3,16α-diol it was only possible to detect 6%.

These test results open up to the compounds of the general formula (I) according to the invention various possibilities of use for hormone replacement therapy (HRT) and in hormonally related diseases in men and women.

The present invention therefore also relates to pharmaceutical compositions which comprise at least one compound of the general formula (I), optionally together with pharmaceutically tolerable excipients and vehicles.

Compared to their parent oestrogens, the substances according to the invention have pharmacodynamically and pharmacokinetically improved properties, which are based on reduced hepatic extraction and more uniform and longer-lasting blood levels of the released oestrogen.

Dosage

For use according to the invention, the ERβ-selective compounds of the general formula (I) are administered orally.

Suitable dosages of the compounds according to the invention to humans for the treatment of peri- and postmenopausal symptoms, of hormone deficiency-related symptoms, of gynaecological disorders such as ovarian dysfunction and endometriosis, of male and female fertility disorders, of hormone-related oncoses and for use in male and female hormone replacement therapy are, depending on indication, 5 μg to 2000 mg per day, depending on age and constitution of the patient, where the necessary daily dose can be administered by single or repeated delivery.

For gynaecological disorders such as ovarian dysfunction and endometriosis, dosages between 0.5 and 100 mg, for the treatment of male and female fertility disorders 5 μg to 50 mg, for hormone-related oncoses 5 to 500 mg and for male or female hormone replacement therapy 5 μg to 100 mg are possible,

In addition to customary vehicles and/or diluents, the pharmaceutical compositions comprise at least one compound of the general formula I. The substances according to the invention can also be employed therapeutically in combination with a gestagen, antigestagen or mesoprogestin. Preferably, the substances according to the invention are administered individually as an active compound in pharmaceutical preparations.

The medicaments of the invention are prepared in a known manner with a suitable dosage using the customary solid or liquid vehicles or diluents and the customarily used pharmaceutical excipients according to the desired type of administration. The preferred preparations consist in an administration form which is suitable for oral administration. Such administration forms are, for example, tablets, film-coated tablets, coated tablets, capsules, pills, powders, solutions or suspensions or alternatively depot forms.

Appropriate tablets can be obtained, for example, by mixing the active compound with known excipients, for example inert diluents such as dextrose, sugar, sorbitol, mannitol, polyvinylpyrrolidone, disintegrants such as maize starch or alginic acid, binders such as starch or gelatine, lubricants such as magnesium stearate or talc and/or agents for achieving a depot effect such as carboxypolyethylene, carboxymethyl-cellulose, cellulose acetate phthalate or polyvinyl acetate. The tablets can also consist of a number of layers.

Correspondingly, coated tablets can be prepared by coating of cores produced analogously to the tablets with agents customarily used in coated tablet coatings, for example polyvinylpyrrolidone or shellac, gum arabic, talc, titanium oxide or sugar. Here, the coated tablet shells can also consist of a number of layers, where the excipients mentioned above in the case of the tablets can be used.

Solutions or suspensions using the compounds of the general formula I according to the invention can additionally comprise taste-enhancing agents such as saccharin, cyclamate or sugar and also, for example, flavourings such as vanillin or orange extract, They can moreover comprise suspending excipients such as sodium carboxymethylcellulose or preservatives such as p-hydroxybenzoates.

The capsules comprising compounds of the general formula I can be prepared, for example, by mixing the compound(s) of the general formula I with an inert carrier such as lactose or sorbitol and encapsulating them in gelatine capsules.

The following examples illustrate the present invention without restricting it.

### EXAMPLE 1

### 3-Hydroxy-9-vinyloestra-1,3,5(10)-trien-16α-yl 3′-sulphamoylbenzoate

Stage 1

3,16α-Bis(tert-butyldimethylsilyloxy)-9α-vinyloestra-1,3,5(10)-triene

1.0 g (3.35 mmol) of 9α-vinyloestra-1r3,5(10)-triene-3,16α-diol and 2.1 g (13.4 mmol) of tert-butyldimethyl-chlorosilane are initially introduced in 25 ml of dimethylformamide and treated in portions with 1.8 g (26.8 mmol) of imidazole with stirring at room temperature. After about 30 minutes, a white suspension is obtained. The reaction solution is poured into ice water with vigorous stirring. The precipitated product is filtered off with suction, washed with water and dried. 1.62 g (92% of theory) of 3,16α-bis(tert-butyl-dimethylsilyloxy)-9α-vinyloestra-1,3,5(10)-triene are obtained.

Stage 2

3-(tert-Butyldimethylsilyloxy)-9α-vinyloestra-1,3,5(10)-trien-16α-ol

0.8 g (1.52 mmol) of 3,16α-bis(tert-butyldimethyl-silyloxy)-9α-vinyloestra-1,3,5(10)-triene is initially introduced in 40 ml of absolute ethanol and treated at room temperature with 1.0 ml (8.0 mmol) of boron trifluoride etherate with stirring and exclusion of moisture. After about 60 minutes, the reaction is ended by addition of sodium hydrogencarbonate solution. Ethanol is distilled off from the reaction mixture and the reaction products are extracted with ethyl acetate. The 3-(tert-butyldimethylsilyloxy)-9α-vinyloestra-1,3,5(10)-trien-16α-ol is purified by column chromatography on silica gel 60 and isolated. 0.59 g (94% of theory) is obtained.

Stage 3

3-(tert-Butyldimethylsilyloxy)-9α-vinyloestra-1,3,5(10)-trien-16α-yl 3′-sulphamoylbenzoate

0.3 g (0.73 mmol) of 3-(tert-butyldimethylsilyloxy)-9α-vinyloestra-1,3,5(10)-trien-17β-ol is dissolved in 2 ml of pyridine and 2 ml of methylene chloride. 0.3 ml (2 mmol) of 3-chlorosulphonylbenzoyl chloride is added to the reaction mixture at −20° C. with stirring. Subsequently, the mixture is warmed to room temperature and stirred for 15 min. 25 ml of conc. ammonia solution are added to the reaction solution and it is stirred intensively for 15 min. The pH is adjusted to 5 using 10% strength hydrochloric acid. The organic solvents are distilled off to the greatest possible extent. The precipitated substance is filtered off with suction and washed with water. 420 mg of 3-(tert-butyldimethyl-silyloxy)-9α-vinyloestra-1,3,5(10)-trien-16α-yl 3′-sulphamoylbenzoate are obtained as a crude product.

Stage 4

3-Hydroxy-9α-vinyloestra-l,3,5(10)-trien-16α-yl 3-sulphamoylbenzoate

420 mg of crude product from Stage 3 are dissolved in 10 ml of tetrahydrofuran. 420 mg (1.3 mmol) of tetra-n-butylammonium fluoride trihydrate are added at room temperature with stirring. After 1 hour, 40 ml of water are stirred in, tetrahydrofuran is distilled off and the reaction product is extracted with ethyl acetate. After isolation of the reaction product by distilling off the ethyl acetate, the crude product is purified by chromatography on silica gel 60. 295 mg of 3-hydroxy-9α-vinyloestra-1,3,5(10)-trien-16α-yl 3′-sulphamoyl-benzoate (84% of theory based on 3-(tert-butyldimethyl-silyloxy)-9α-vinyloestra-1,3,5(10)-trien-17β-ol) are obtained.

¹H—NNR (400 MHz, DMSO-d₆, TMS): 9.00 (s, 3-OH); 8.39 (s, 1H); 8.16 (m, 1H); 8.08 (m, 1H); 7.73 (m, 1H); 7.55 (s, 2H, NH₂); 7.00 (d, J=8.6 Hz, H-1; 6.53 (dd, J=8.6/2.7 Hz, H-2); 6.43 (d, J=2.7 Hz, H-4); 6.28 (dd, J=17.2/10.5 Hz, —CH=CH₂); 5.44 (m, 1H, H-16β); 5.02 (dd, J=10.5/1.9 Hz, —CH=CH₂); 4.48 (dd, J=17.2/1.9 Hz, —CH=CH₂); 0.82 (s, 3H, H-18).

### EXAMPLE 2

### 3-Hydroxy-17β-fluoro-9-vinyloestra-1,3,5(10)-trien-17β-yl 31-sulphamoylbenzoate

Stage 1

3, 16α-Bis (tert-butyldimethylsilyloxy)-17β-fluoro-9α-vinyloestra-1,3,5(10)-triene

1.0 g (3.16 mmol) of 9α-vinyloestra-1,3,5(10)-triene-3,16α-diol and 2.1 g (13.4 mmol) of tert-butyldimethyl-chlorosilane are initially introduced in 25 ml of dimethylformamide and treated in portions with 1.8 g (26.8 mmol) of imidazole with stirring at room temperature. After about 30 minutes, a white suspension is obtained. The reaction solution is poured into ice water with vigorous stirring. The precipitated product is filtered off with suction, washed with water and dried. 1.53 g (87% of theory) of 3,16α-bis(tert-butyl-dimethylsilyloxy)-17β-fluoro-9α-vinyloestra-1,3,5(10)-triene are obtained.

Stage 2

3-(tert-Butyldimethylsilyloxy)-17β-fluoro-9α-vinyl-oestra-1,3,5(10)-trien-16α-ol

1.53 g (2.81 mmol) of 3,16α-bis(tert-butyldimethyl-silyloxy)-17β-fluoro-9α-vinyloestra-1,3,5(10)-triene are initially introduced in 40 ml of absolute ethanol and treated at room temperature with 1.8 ml (14.8 mmol) of boron trifluoride etherate with stirring and exclusion of moisture. After about 60 minutes, the reaction is ended by addition of sodium hydrogen-carbonate solution, Ethanol is distilled off from the reaction mixture and the reaction products are extracted with ethyl acetate. The 3-(tert-butyl-dimethylsilyloxy)-17β-fluoro-9α-vinyloestra-1,3,5(10)-trien-16α-ol is purified by column chromatography on silica gel 60 and isolated by distilling off the eluent. 1.21 g (91% of theory) are obtained.

Stage 3

3-(tert-Butyldimethylsilyloxy)-17β-fluoro-9α-vinyl-oestra-1,3,5(10)-trien-16α-yl 3′-sulphamoylbenzoate

1.21 g (2.81 mmol) of 3-(tert-butyldimethylsilyloxy)-17β-fluoro-9α-vinyloestra-1,3,5(10)-trien-17β-ol are dissolved in 8 ml of pyridine and 8 ml of methylene chloride. 1.2 ml (8.0 mmol) of 3-chlorosulphonylbenzoyl chloride are added to the reaction mixture at −20° C. with stirring. Subsequently, the mixture is warmed to room temperature and stirred for 15 min. 25 ml of conc. ammonia solution are added to the reaction solution and it is stirred intensively for 15 min. The pH is adjusted to 5 using 10% strength hydrochloric acid. The organic solvents are distilled off to the greatest possible extent. After separating off, the precipitated substance is washed with water. 1.62 g of 3-(tert-butyldimethylsilyloxy)-17β-fluoro-9α-vinyloestra-1,3,5(10)-trien-16α-yl 3′-sulphamoylbenzoate are obtained as an oily crude product.

Stage 4

3-Hydroxy-17β-fluoro-9α-vinyloestra-1,3,5(10)-trien-16α-yl 3,-sulphamoylbenzoate

1.62 g of crude product from Stage 3 are dissolved in 30 ml of tetrahydrofuran. 1.62 g (5.0 mmol) of tetra-n-butylammonium fluoride trihydrate are added at room temperature with stirring. After 1 hour, 40 ml of water are stirred in, tetrahydrofuran is distilled off and the reaction product is extracted with ethyl acetate. After isolation of the reaction product by distilling off the ethyl acetate, the crude product is purified by chromatography on silica gel 60. 1.14 g of 3-hydroxy-17β-fluoro-9α-vinyloestra-1,3,5(10)-trien-17α-yl 3-sulphamoylbenzoate (81% of theory based on 3-(tert-butyldimethylsilyloxy)-17β-fluoro-9α-vinyloestra-1,3,5(10)-trien-17β-ol) are obtained.

¹H—NMR (400 MHz, DMSO-d₆, TMS): 9.07 (s, 3-OH); 8.42 (s, 1H); 8.22 (d, J=7.8 Hz, 1H); 8.09 (d, J=7.8 Hz, 1H); 7.76 (t, J=7.8 Hz, 1H); 7.56 (s, 2H, NH₂); 7.00 (d, J=8.6 Hz, H-1); 6.52 (dd, J=8.6/2.7 Hz, H-2); 6.42 (d, J=2.4 Hz, H-4); 6.29 (dd, J=17.2/10.7 Hz, CH=CH₂); 5.39 (m, 1H, H-16β); 5.03 (dd, J=10.7/1.9 Hz, —CH=CH₂); 4.95 (dd, J=54.3/5.1 Hz, H-17α); 4.45 (dd, J=17.2/1.9 Hz, —CH=CH₂); 0.92 (d, J=1.0 Hz, 3H, H-18).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102005057224.3, filed Nov. 29, 2005, and U.S. Provisional Application Ser. No. 60/742,524, filed Dec. 6, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### REFERENCES

1. Cummings SR, Browner WS, Bauer D, Stone K, Ensrud K, Jamal S and Ettinger B (1998), Endogenous hormones and the risk of hip and vertebral fractures among older women. N. Engl. J. Med. 339, 733-38.
2. Frank G R (1995), The role of estrogen in pubertal skeletal physiology: epiphyseal maturation and mineralization of the skeleton. Acta Paediatr. 84(6), 627-30.
3. Coldzieher J W (1990), Selected aspects of the pharmacokinetics and metabolism of ethinyl estrogens and their clinical implications. Am. J. Obstet. Gynecol. 163, 318-22.
4. Gustafsson J A (2000), Novel aspects of estrogen action. J. Soc. Gynecol. Investig. 7, S8-S9.
5. Helmer O M and Griffith R S (1952), The effect of the administration of estrogens on the renin-substrate (hypertensinogen) content on rat plasma. Endocrinology 51, 421-6.
6. Kelly J J, Rajkovic I A, O'Sullivan A J, Sernia C and Ho K K Y (1993), Effects of different oral oestrogen formulations on insulin-like growth factor-I, growth hormone and growth hormone binding protein in post-menopausal women. Clin. Endocrinol. 39, 561-67.
7. Krattenmacher R, Knauthe R, Parczyk K, Walker A, Hilgenfeldt U and Fritzemeier K-H (1994), Estrogen action on hepatic synthesis of angiotensinogen and IGF-I: direct and indirect estrogen effects. J. Steroid. Biochem. Mol. Biol. 48, 207-14.
8. Le Roith and Butler A A (1999), Insulin-like growth factors in pediatric health and disease. J. Clin. Endocrinol. Metab. 84, 4355-61.
9. Mandel F P, Geola F L, Lu J K H, Eggena P, Sambhi M P, Hershman J M and Judd H L (1982), Biologic effects of various doses of ethinyl estradiol in postmenopausal women. Obstet. Gynecol. 59, 673-9.
10.Mashchak C A, Lobo R A, Dozono-Takano R, Eggena P, Nakamura R M, Brenner P F and Mishell D R Jr (1982), Comparison of pharmacodynamic properties of various estrogen formulations. Am. J. Obstet. Gynecol. 144, 511-18.
11.Oelkers W K H (1996), Effects of estrogens and progestagens on the renin-aldosterone system and blood pressure. Steroids 61, 166-71.
12. O'Sullivan A J and Ho K K Y (1995), A comparison of the effects of oral and transdermal estrogen replacement on insulin sensitivity in postmenopausal women. J. Clin. Endocrinol. Metab. 80, 1783-8.
13. Span J P T, Pieters G F F M, Sweep C G J, Hermus A R M M and Smals A G H (2000), Gender difference in insulin-like growth factor I response to growth hormone (GH) treatment in GH-deficient adults: role of sex hormone replacement. J. Clin. Endocrinol. Metab. 85, 1121-5.
14.von Schoultz B, Carlström K, Collste L, Eriksson A, Henriksson P, Pousette A and Stege R (1989), Estrogen therapy and liver function—metabolic effects of oral and parenteral administration. Prostate 14, 389-95.

1. Sulphamoyl compounds of 9α-substituted oestra-trienes of the general formula (I) 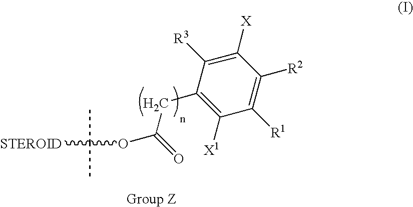
in which n is a number 0-4,
R¹ is a radical —SO₂NH₂ or —NHSO₂NH₂,
where R², R³ and X, X¹ independently of one another are a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
where R²⁰ and R²¹ are a C₁₋₅-alkyl group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
R²⁰ can moreover be a hydrogen, or
R² is a radical —SO₂NH₂ or —NHSO₂NH₂,
where R¹, R³ and X, X¹ independently of one another are a hydrogen atom, a halogen atom, a nitrile group, a nitro group, a C₁₋₅-alkyl group, a CₚF₂ₚ₊group with p=1-3, a group OC(O)—R²⁰, COOR²⁰, OR²⁰, C(O)NHR²⁰ or OC(O)NH—R²¹,
where R²⁰ and R²¹ are a C₁₋₅-alkyl group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
R²⁰ can moreover be a hydrogen, or
R³ is a radical —SO₂NH₂ or —NHSO₂NH₂,
where R¹, R² and X, X¹ independently of one another are a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
where R²⁰ and R²¹ are a C₁₋₅-alkyl group, a C₃₋₈-cycloalkyl group, an aryl group, a C₁₋₄-alkylenearyl group, a C₁₋₄-alkylene-C₃₋₈-cyclo-alkyl group or C₃₋₈-cycloalkylene-C₁₋₄-alkyl group, and
R²⁰ can moreover be a hydrogen, and
STEROID is a steroidal ABCD ring system of the formula (A): 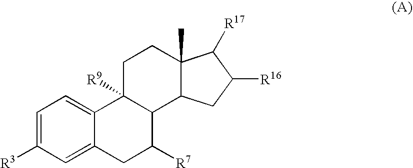
where the radicals R⁷, R⁹, R¹⁶ and R¹⁷ have the following meaning:
R³ is Z and
R¹⁶ is an OH group, a tri(C₁₋₄-alkyl)silyloxy group or a group OC(O)—R^{20 , or}
R³ is OH, OMe, a tri(C₁₋₄-alkyl)silyloxy group, a group OC(O)—R²⁰ and
R¹⁶ is Z and
R⁷ is a hydrogen atom or fluorine atom, a methyl radical or ethyl radical,
R⁹ is a branched or straight-chain, optionally partially or completely halogenated alkyl, alkenyl or alkynyl radical having up to 3 carbon atoms,
R¹⁷ is a hydrogen atom or a halogen atom
where the substituents R⁷, R¹⁶ and R¹⁷ can in each case be both in the α-position and in the β-position,
and their pharmaceutically acceptable salts.

2. Compounds according to claim 1, characterized in that n is 0, 1 or 2.

3. Compounds according to claim 1, characterized in that in each case one radical R¹, R² or R³ is a group —SO₂NH₂.

4. Compounds according to claim 1, characterized in that R¹ is a group —SO₂NH₂ or —NHSO₂NH₂.

5. Compounds according to claim 4, characterized in that R¹ is a group —SO₂NH₂.

6. Compounds according to claim 1, characterized in that if one of the radicals R¹, R², R³ is not —SO₂NH₂ or —NHSO₂NH₂, the other two radicals of R¹, R², R³ and X and X¹ in each case independently of one another are a hydrogen, fluorine or chlorine atom, or a hydroxyl or a methoxy group.

7. Compounds according to claim 1, characterized in that R⁶ is a methyl, ethyl, vinyl or difluorovinyl radical.

8. Compounds according to claim 1,
1) 3-hydroxy-9α-vinyloestra-1,3,5(10)-trien-16α-yl 3′-sulphamoylbenzoate,
2) 3-hydroxy-17β-fluoro-9α-vinyloestra-1,3,5(10)-trien-16α-yl 3′-sulphamoylbenzoate,
3) 3-hydroxy-7α-methyl-9α-vinyloestra-1,3,5(10)-trien-16α-yl 3′-sulphamoylbenzoate,
4) 3-hydroxy-7α-fluoro-9α-vinyloestra-1,3,5(10)-trien-16α-yl 3′-sulphamoylbenzoate,
5) 3-acetoxy-9α-vinyloestra-1,3,5(10)-trien-16α-yl 3′-sulphamoylbenzoate,
6) 3-acetoxy-17β-fluoro-9α-vinyloestra-1,3,5(10)-trien-16α-yl 3′-sulphamoylbenzoate,
7) 3-acetoxy-7α-methyl-9α-vinyloestra-1,3,5(10)-trien-16α-yl 3′-sulphamoylbenzoate,
8) 3-acetoxy-7α-fluoro-9α-vinyloestra-1,3,5(10)-trien-16α-yl 3′-sulphamoylbenzoate,
9) 3-hydroxy-9α-vinyloestra-1,3,5(10)-trien-16α-yl 2′-chloro-5 ′-sulphamoylbenzoate,
10) 3-hydroxy-17β-fluoro-9α-vinyloestra-1,3,5(10)-trien-16α-yl 2′-chloro-5′-sulphamoylbenzoate,
11) 3-hydroxy-7α-methyl-9α-vinyloestra-1,3,5(10)-trien-16α-yl 2′-chloro-5′-sulphamoylbenzoate,
12) 3-hydroxy-7α-fluoro-9α-vinyloestra-1,3,5(10)-trien-16α-yl 2′-chloro-5′-sulphamoylbenzoate,
13) 3-acetoxy-9α-vinyloestra-1,3,5(10)-trien-16α-yl 2′-chloro-5′-sulphamoylbenzoate,
14) 3-acetoxy-17β-fluoro-9α-vinyloestra-1,3,5(10)-trien-16α-yl 2′-chloro-5′-sulphamoylbenzoate,
15) 3-acetoxy-7α-methyl-9α-vinyloestra-1,3,5(10)-trien-16α-yl 2′-chloro-5′-sulphamoylbenzoate,
16) 3-acetoxy-7α-fluoro-9α-vinyloestra-1,3,5(10)-trien-16α-yl 2′-chloro-5′-sulphamoylbenzoate,
17) 3-hydroxy-9α-vinyloestra-1,3,5(10)-trien-16α-yl 4′-sulphamoylbenzoate,
18) 3-hydroxy-17β-fluoro-9α-vinyloestra-1,3,5(10)-trien-16α-yl 4′-sulphamoylbenzoate,
19) 3-hydroxy-7α-methyl-9α-vinyloestra-1,3,5(10)-trien-16α-yl 4′-sulphamoylbenzoate,
20) 3-hydroxy-7α-fluoro-9α-vinyloestra-1,3,5(10)-trien-16α-yl 4′-sulphamoylbenzoate,
21) 3-acetoxy-9α-vinyloestra-1,3,5(10)-trien-16α-yl 4′-sulphamoylbenzoate,
22) 3-acetoxy-17β-fluoro-9α-vinyloestra-1,3,5(10)-trien-16α-yl 4′-sulphamoylbenzoate,
23) 3-acetoxy-7α-methyl-9α-vinyloestra-1,3,5(10)-trien-16α-yl 4′-sulphamoylbenzoate,
24) 3-acetoxy-7α-fluoro-9α-vinyloestra-1,3,5(10)-trien-16α-yl 4′-sulphamoylbenzoate.

9. Pharmaceutical compositions comprising at least one compound according to claim 1, and a pharmaceutically tolerable carrier.

10. Pharmaceutical composition according to claim 9, characterized in that at least one further steroidal compound is present.

11. Pharmaceutical composition according to claim 10, characterized in that the further steroidal compound is a gestagen, antigestagen or mesoprogestin.

12. Pharmaceutical composition according to claim 11, where the gestagen is drospirenone, dienogest, norethisterone or levonorgestrel, and the anti-gestagen is onapristone or mifepristone or the mesoprogestin is asoprisnil.

13. Use of the compounds according to the invention as set forth in claim 1 for the production of a medicament.

14. Use according to claim 13 for the treatment of diseases and conditions in women and in men which are caused by an oestrogen deficit.

15. Use according to claim 13 for the treatment of peri- and postandropausal symptoms.

16. Use according to claim 13 for the in vitro treatment of male infertility.

17. Use according to claim 13 for the in vivo treatment of male infertility.

18. Use according to claim 13 for the in vitro treatment of female infertility.

19. Use according to claim 13 for the in vivo treatment of female infertility.

20. Use according to claim 13 for the therapy of hormone deficiency-related symptoms in ovarian dysfunction caused surgically, medicinally or in another way.

21. Use according to claim 13 for hormone replacement therapy (HRT).

22. Use according to claim 20 in combination with a selective oestrogen receptor modulator (SERM), for example raloxifen.

23. Use according to claim 13 for the prophylaxis and therapy of a hormone deficiency-related loss of bone mass.

24. Use according to claim 13 for the prophylaxis and therapy of osteoporosis.

25. Use according to claim 23 in combination with the natural vitamin D3 or with calcitriol analogues for osteogenesis or as a supportive therapy for therapies which cause a loss of bone mass (for example a therapy using glucocorticoids, aromatase inhibitors, GnRH agonists or antagonists, chemotherapy).

26. Use according to claim 13 for the prevention and therapy of cardiovascular diseases.

27. Use according to claim 13 for the treatment of inflammatory diseases and diseases of the immune system.

28. Use according to claim 27 for the treatment of rheumatoid arthritis.

29. Use according to claim 27 for the treatment of multiple sclerosis, Crohn's disease or endometriosis.

30. Use according to claim 13 for the prevention and treatment of benign prostate hyperplasia (BPH).

31. Use according to claim 30 in combination with anti-oestrogens and selective oestrogen receptor modulators for the prevention and treatment of benign prostate hyperplasia (BPH).

32. Use according to claim 31, where the anti-oestrogen used is 7α-[9-[(4,4,5,5,5-pentafluoro-pentyl)sulphinyl]nonyl]oestra-1,3,5(10)-triene-3,17β-diol (fulvestrant) and the SERM used is raloxifen, tamoxifen or 5-(4-{5-[(RS)-(4,4,5,5,5-pentafluoropentyl)sulphinyl)pentyl}phenyl)-6-phenyl-8,9-dihydro-7H-benzocyclohepten-2-ol.

33. Use according to claim 13 for the treatment of arthritic symptoms, in particular after therapies which lead to oestrogen deprivation, for example after treatment with aromatase inhibitors or GnRH antagonists or agonists.

34. Use of compounds according to claim 1 for production of medicaments for the treatment of diseases which can be positively influenced by the inhibition of the carboanhydrase activity.

35. Use of compounds according to claim 1 for production of medicaments for the treatment of alopecia.

36. Use of 3-hydroxy-9α-vinyloestra-1,3,5(10)-trien-16β-yl 3′-sulphamoylbenzoate, 3-acetoxy-9α-vinyl-oestra-1,3,5(10)-trien-16β-yl 3′-sulphamoyl-benzoate as set forth in claim 13.

37. Process for the preparation of compounds of the general formula (I) according to claim 1 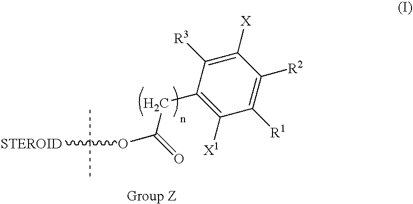
by reaction of 9α-substituted oestratrienes as set forth in formula (A) with appropriate sulphamoyl-phenylcarboxylic acids or their derivatives or by reaction of appropriate compounds with sulphamide, sulphamoyl chloride or aminosulphonyl isocyanate.
